(19) United States
(12) Patent Application Publication
(10) Pub. No.: US 2024/0256823 A1
(43) Pub. Date: Aug. 1, 2024

(54) SYSTEM AND METHOD FOR IDENTIFYING ONE OR MORE CHANGES IN BIOLOGICAL NETWORK

(71) Applicant: Innoplexus AG, Eschborn (DE)

(72) Inventors: Vinay Chandil, Gwalior (IN); Pooja Chaskar, Pimpri-Chinchwad (IN); Om Sharma, Pimpri Chinchwad (IN)

(73) Assignee: Innoplexus AG, Eschborn (DE)

(21) Appl. No.: 18/161,392

(22) Filed: Jan. 30, 2023

(51) Int. Cl. G06N 3/00

(52) U.S. Cl. CPC G06N 3/002

(57) ABSTRACT

A system for identifying one or more changes in a Biological Network, comprising a processor configured to construct a Heterogeneous Biological Network that comprises a plurality of nodes and a plurality of edges. The processor is configured to derive one or more sub-networks from the constructed HBN, and determine an embedding vector for each node of each sub-network. The processor is configured to identify one or more changes in each sub-network by comparing the embedding vector of each node of in a respective sub-network before and after an input action associated with a change in at least one sub-network and determine a plurality of scores for each node of each sub-network based on a pre-defined set of parameters. The processor is configured to identify the one or more changes in the BN based on the determined plurality of scores for each node in each sub-network.

### TECHNICAL FIELD

The aspects of the disclosed embodiments relate generally to the field of biological networks; and more specifically, to a system and a method for identifying and quantifying one or more changes in a biological network.

### BACKGROUND

With the continuous discovery of various biological entities and their molecular relationships, large Heterogeneous Biological Networks (HBN) are created. The molecular relationships among the various biological entities are primary keys to discover underlying behavioural mechanisms of the biological entities and develop therapeutics, of which early steps are to develop hypotheses where, analysing the large HBN is required to obtain high-detailed information about the large HBN. Although analysing and gathering insights about the large and complex HBN is a technical challenge. The mere visualization and storage of different types of biological entities in the large HBN does not provide the information relevant to the biological entities which are indirectly correlation with each other. Moreover, the several obvious behaviours are hidden inside the large HBN which, are typically determined by cost-intensive, time-consuming and resource consuming processes.

Currently, certain attempts have been made to analyse and gather insights of a conventional HBN, such as use of a methodology related to topological analysis of the conventional HBN. The methodology includes centrality (i.e., degree, or closeness, or betweenness, or eigen, and the like) where information carried for a node is only limited to the number of edges and the number of neighbouring nodes. However, the depth level information about the topology of the conventional HBN is often missing in this methodology. Other methodologies based on certain embeddings use a Deep Neural Network (DNN) to find associations between various biological entities of the conventional HBN. These methodologies use lower dimensional embeddings to optimize computations hence, in case a large number of nodes and edges exist in the conventional HBN, these methodologies fail to capture the complete information on a node. Thus, there exists a technical problem of how to quantify the flow of information and changes between various biological entities of the conventional HBN and thereby, understanding the dynamics of disease conditions, therapeutic responses and lead identifications.

Therefore, in light of the foregoing discussion, there exists a need to overcome the aforementioned drawbacks associated with the conventional methods of analysing and gathering insights of a large and complex HBN.

### SUMMARY

The aspects of the disclosed embodiments are directed to a system and a method for identifying and quantifying one or more changes in a Biological Network. An aim of the disclosed embodiments is to provide an improved system and an improved method for identifying one or more changes in a biological network.

One or more advantages of the disclosed embodiments are achieved by the solutions provided in the enclosed independent claims. Advantageous implementations of the present disclosure are further defined in the dependent claims.

In an aspect, the aspects of the disclosed embodiments provide a system for identifying one or more changes in a Biological Network (BN). The system comprises a processor configured to construct a Heterogeneous Biological Network (HBN) that comprises a plurality of nodes and a plurality of edges. The processor is further configured to derive one or more sub-networks from the constructed HBN, wherein each sub-network comprises a set of nodes of the plurality of nodes and a set of edges of the plurality of edges of the constructed HBN and determine an embedding vector for each node of the set of nodes of each sub-network, wherein the embedding vector represents a topology and a plurality of connections of each node in a respective sub-network. The processor is further configured to identify one or more changes in each sub-network by comparing the embedding vector of each node of the set of nodes in the respective sub-network before and after an input action associated with a change in at least one sub-network and determine a plurality of scores for each node of the set of nodes of each sub-network based on a pre-defined set of parameters. The processor is further configured to identify the one or more changes in the BN based on the determined plurality of scores for each node of the set of nodes in each sub-network, wherein the plurality of scores is processed at each sub-network and at the BN for identification of the one or more changes in the BN.

The system efficiently quantifies the flow of information and the one or more changes among the number of biological entities of the BN and thereby, improves the prediction of disease conditions, therapeutic responses and lead identifications. For quantification of the changes of the BN, the system is configured to compare and analyse different hypotheses related to therapeutic responses and disease mechanisms. The system is configured to use graph theory and database technologies to process, analyse and visualise the BN and derive deeper insights (i.e., indirect relationships among biological entities) using graph projections, embedding vectors and statistical solutions. By virtue of using the graph projections, embedding vectors and statistical solutions, the system is configured to measure the network mutations score of the BN which, become a basis to accept or to negate the hypotheses. The network mutations may also be compared with each other across different hypotheses to check which hypothesis is better. Moreover, the system can be customized according to the nature of the hypothesis. Conventionally, betweenness centrality is used to calculate unweighted shortest paths between all pairs of nodes in a biological network. Each node receives a score based on the number of shortest paths that pass through the node. The nodes lying more frequently on the shortest paths between other nodes have higher betweenness centrality scores. Hence, the node significance is computed by considering only the network structure. However, the system is configured to use the network structure, such as change in the node embeddings before and after removing nodes, etc. as well as consider other parameters, such as publication count, Lagrange Multiplier (LM) score and aggregation score of each node, which indicate the functionality of the node. Thus, the system considers both the network structure as well as the functionality of the node which overcome the limitations of the betweenness centrality.

In another aspect, the aspects of the disclosed embodiments provide a method for identifying one or more changes in a Biological Network (BN). The method comprises constructing, by a processor, a Heterogeneous Biological Network (HBN) that comprises a plurality of nodes and a plurality of edges. The method further comprises deriving, by the processor, one or more sub-networks from the constructed HBN, wherein each sub-network comprises a set of nodes of the plurality of nodes and a set of edges of the plurality of edges of the constructed HBN and determining, by the processor, an embedding vector for each node of the set of nodes of each sub-network, wherein the embedding vector represents a topology and a plurality of connections of each node in a respective sub-network. The method further comprises identifying, by the processor, one or more changes in each sub-network by comparing the embedding vector of each node of the set of nodes in the respective sub-network before and after an input action associated with a change in at least one sub-network and determining, by the processor, a plurality of scores for each node of the set of nodes of each sub-network based on a pre-defined set of parameters. The method further comprises identifying, by the processor, the one or more changes in the BN based on the determined plurality of scores for each node of the set of nodes in each sub-network, wherein the plurality of scores is processed at each sub-network and at the BN for identification of the one or more changes in the BN.

The method achieves all the advantages and technical effects of the disclosed system of the present disclosure.

It is to be appreciated that all the aforementioned implementation forms can be combined. It has to be noted that all devices, elements, circuitry, units and means described in the present application could be implemented in the software or hardware elements or any kind of combination thereof. All steps which are performed by the various entities described in the present application as well as the functionalities described to be performed by the various entities are intended to mean that the respective entity is adapted to or configured to perform the respective steps and functionalities. Even if, in the following description of specific embodiments, a specific functionality or step to be performed by external entities is not reflected in the description of a specific detailed element of that entity which performs that specific step or functionality, it should be clear for a skilled person that these methods and functionalities can be implemented in respective software or hardware elements, or any kind of combination thereof. It will be appreciated that features of the present disclosure are susceptible to being combined in various combinations without departing from the scope of the present disclosure as defined by the appended claims.

Additional aspects, advantages, features and objects of the present disclosure would be made apparent from the drawings and the detailed description of the illustrative implementations construed in conjunction with the appended claims that follow.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a block diagram of a system for identifying one or more changes in a biological network, in accordance with an embodiment of the present disclosure;

FIGS. 2A to 2C collectively is a flowchart of a method for identifying one or more changes in a biological network, in accordance with an embodiment of the present disclosure;

FIG. 3 is a flowchart to identify network mutations in a heterogeneous biological network, in accordance with an embodiment of the present disclosure;

FIG. 4 illustrates derivation of one or more sub-networks from a heterogeneous biological network, in accordance with an embodiment of the present disclosure;

FIG. 5 illustrates transformation of a heterogeneous biological network into an embedding space, in accordance with an embodiment of the present disclosure;

FIG. 6 is a graphical representation that depicts average running time to generate node embeddings of different lengths, in accordance with an embodiment of the present disclosure; and

FIG. 7 is a bar graph representation that depicts number of embeddings versus count of embeddings having all zero elements, in accordance with an embodiment of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

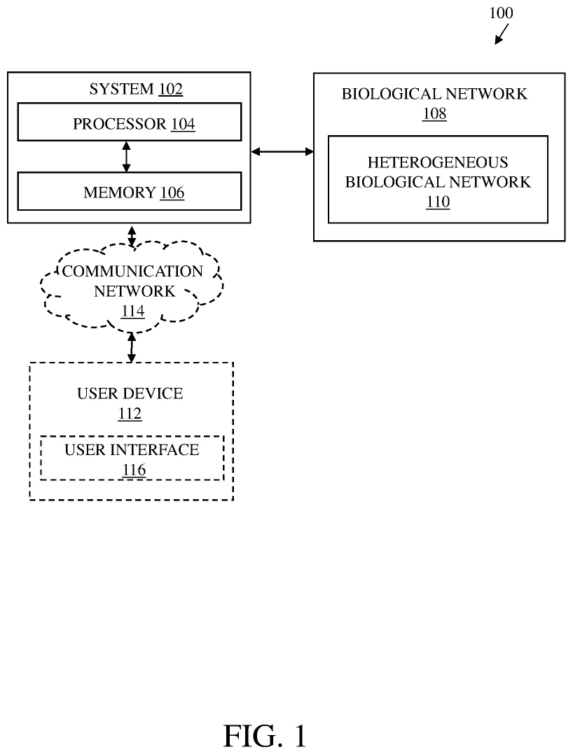

FIG. 1 is a block diagram of a system for identifying one or more changes in a biological network, in accordance with an embodiment of the present disclosure. With reference to FIG. 1, there is shown a block diagram 100 of a system 102 that includes a processor 104 communicably coupled to a memory 106. The system 102 is configured to identify one or more changes in a Biological Network (BN) 108 that comprises a Heterogeneous Biological Network (HBN) 110. Optionally, the system 102 is connected to a user device 112 through a communication network 114. The user device 112 comprises a user interface 116.

The system 102 is configured for identifying one or more changes in the BN 108 and more specifically, in the HBN 110. The BN 108 is widely used in many branches of biology as a convenient representation of various patterns of interactions between appropriate biological elements. The BN 108 is used to capture relationships between various biological entities and objects. A typical representation of the BN 108 consists of a plurality of nodes and a plurality of edges. The BN 108 is of three types: a homogeneous biological network, a heterogeneous biological network (e.g., the HBN 110), and a heterogeneous multi-layered network. The homogeneous biological network is a biological network where all the nodes are identical which means all the nodes have the same relationship in the network, for example, a Protein-Protein Interaction (PPI) network. The HBN 110 consists of two or more different types of nodes having different relationships, for example, a target-pathway association network. In the HBN 110, a node represents a biological entity, such as protein, genes, biological or molecular pathway, disease and conditions, phenotypes, cell lines and tissues, mutations, metabolites, drugs, and the like. The links (or edges) between the two or more different types of nodes represent the relationships among the two or more different types of nodes. For example, a total of 36 types of relationships can be built among the various nodes. By virtue of comprising the different types of nodes and their relationships, the HBN 110 is very large and complex in nature. Conventionally, it is difficult to analyse and gather insights about a typical BN. The mere visualization and storage of different types of biological entities in the typical BN does not provide the information relevant to the biological entities which are indirectly in correlation with each other. In the present disclosure, the system 102 identifies any change that happens in the BN 108 and further quantifies that change as well by virtue of using a graph theory and various database technologies. Alternatively stated, the system 102 identifies the complex and indirect relationships between the various biological entities with an enhanced efficiency and reliability.

The processor 104 may include suitable logic, circuitry, and/or interfaces that is configured to respond and process the instructions required to drive the system 102. Furthermore, the processor 104 may refer to one or more individual processors, processing devices and various elements associated with a processing device that may be shared by other processing devices. Additionally, the one or more individual processors, processing devices and elements are arranged in various architectures for responding to and processing the instructions to drive the system 102. In an implementation, the processor 104 may be an independent unit and located outside the system 102. Examples of the processor 104 may include, but are not limited to a hardware processor, a digital signal processor (DSP), a microprocessor, a microcontroller, a complex instruction set computing (CISC) processor, an application-specific integrated circuit (ASIC) processor, a reduced instruction set (RISC) processor, a very long instruction word (VLIW) processor, a state machine, a data processing unit, a graphics processing unit (GPU), and other processors or control circuitry.

The memory 106 may include suitable logic, circuitry, and/or interfaces that is configured to store data and the instructions executable by the processor 104. Examples of implementation of the memory 106 may include, but are not limited to, an Electrically Erasable Programmable Read-Only Memory (EEPROM), Random Access Memory (RAM), Read Only Memory (ROM), Hard Disk Drive (HDD), Flash memory, Solid-State Drive (SSD), or CPU cache memory. The memory 106 may store an operating system or other program products (including one or more operation algorithms) to operate the system 102.

The communication network 114 may include suitable logic, circuitry, and/or interfaces through which the system 102 is connected to the user device 112. Examples of implementation of the communication network 114 may include, but are not limited to, a cellular network (e.g., a 2G, a 3G, long-term evolution (LTE) 4G, a 5G, or 5G NR network, such as sub 6 GHz, cmWave, or mmWave communication network), a wireless sensor network (WSN), a cloud network, a Local Area Network (LAN), a vehicle-to-network (V2N) network, a Metropolitan Area Network (MAN), and/or the Internet.

The user device 112 may include suitable logic, circuitry, and/or interfaces that is used by a user (not shown in FIG. 1) for analyzing the identified one or more changes in the BN 108. The user device 112 comprises the user interface 116 which displays the identified one or more changes in the BN 108 to the user. Examples of implementation of the user device 112 may include, but are not limited to, a computer, mobile phone, laptop, a display device, and the like.

In operation, the aspects of the disclosed embodiments provide the system 102 for identifying one or more changes in the biological network 108. The system 102 comprises the processor 104 configured to construct the HBN 110 that comprises a plurality of nodes and a plurality of edges. The system 102 is used to quantify the information in the BN 108 and identify the various network mutations in the BN 108 using graph theory and database technologies. The network mutations may be defined as the combination of topological and behavioural changes in the BN 108. The identification of the network mutations in the BN 108 is required to improve the understanding of dynamics of disease conditions, therapeutic responses and lead identifications. Moreover, a network mutation index in the BN 108 supports the decisions about hypotheses which, are based on complex inter-connections of the plurality of nodes with each other. Moreover, the plurality of nodes of the constructed HBN 110 represent a number of biological entities, such as protein, genes, target, drug, pathway, diseases, and the like. The plurality of edges of the constructed HBN 110 represent a number of relationships among the number of biological entities, such as activation, inhibition between the biological entities, and the like. Additionally, the information carried by the constructed HBN 110 is stored in a graphical representation for example, by use of a neo4j technology. The graphical representation of the constructed HBN 110 may also be referred to as a meta-graph.

In accordance with an embodiment, the processor 104 is further configured to construct the HBN 110 using one of: a manual approach, curated database, statistical approach, inferred relationships approach, link prediction approach, and data extraction approach. The processor 104 is configured to construct the HBN 110 using either the manual approach (i.e., by reading articles or curation), or the curated database (i.e., using database, such as DrugBank, and the like), or the statistical approach (i.e., overlap between common factors, such as hypergeometric approach), or the inferred relationship approach (i.e., from network “a” connected to “b”, “b” connected to “c”. “a” is connected to “c” which, is the inferred relationship), or the link prediction approach (i.e., use of machine learning or deep learning methods), or the data extraction approach (i.e., text processing).

The processor 104 is further configured to derive one or more sub-networks from the constructed HBN 110, wherein each sub-network comprises a set of nodes of the plurality of nodes and a set of edges of the plurality of edges of the constructed HBN 110. The one or more sub-networks are a part of the constructed HBN 110 which are derived by focusing on the type of nodes and relationships types of interest. The one or more sub-networks (or sub-network projections) from the constructed HBN 110 are required to analyse the direct or indirect relationships among various biological entities in detail. Moreover, the one or more sub-networks are used to perform a fast and precise analysis depending on a use case and derive insights relevant to a focused hypothesis. Since, each sub-network is a part of the constructed HBN 110 therefore, each sub-network comprises the set of nodes of the plurality of nodes and the set of edges of the plurality of edges of the constructed HBN 110.

In accordance with an embodiment, the one or more sub-networks comprises a homogeneous network, a heterogeneous network, a heterogeneous multi-layered network, or a combination thereof. The one or more sub-networks derived from the constructed HBN 110 may include the homogeneous network (e.g., a Protein-Protein Interaction, PPI network), the heterogeneous network (e.g., a Protein-Pathway Interaction network), the heterogeneous multi-layered network (e.g., a Protein-Pathway-Disease network) or a combination of aforementioned networks depending on an application scenario.

The processor 104 is further configured to determine an embedding vector for each node of the set of nodes of each sub-network, wherein the embedding vector represents a topology and a plurality of connections of each node in a respective sub-network. After derivation of the one or more sub-networks from the constructed HBN 110, the information about each node of the set of nodes of each sub-network is stored in a high-dimensional embedding space by creating a vector which represents the topology and the plurality of connections of each node in the respective sub-network.

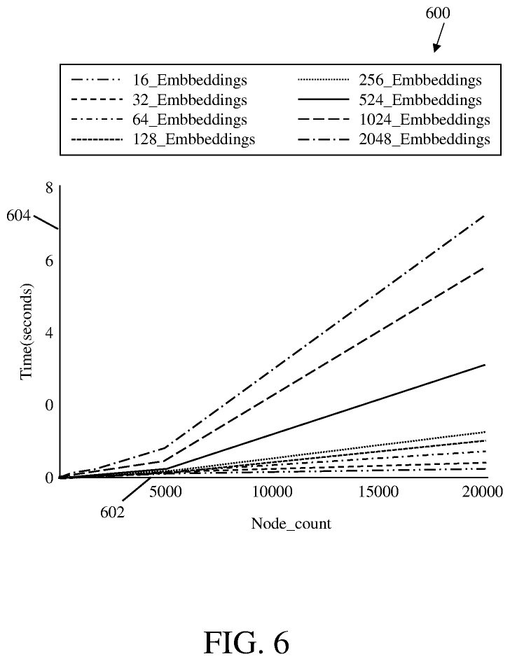

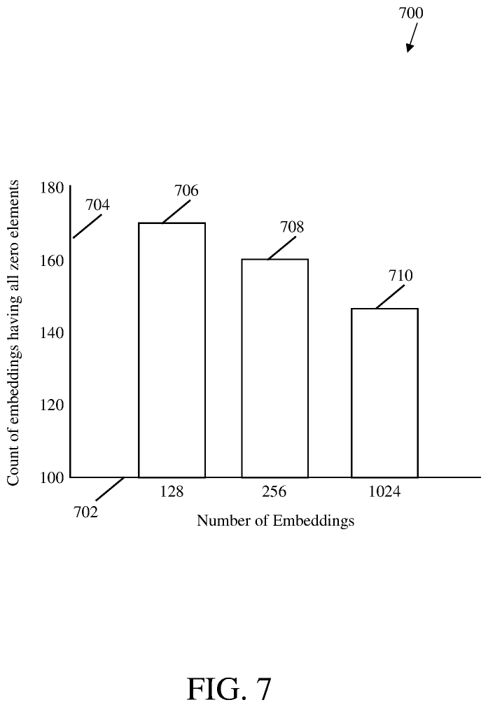

In accordance with an embodiment, the processor 104 is further configured to determine a dimension size of the embedding vector for each node in each sub-network. The determination of the dimension size of the embedding vector for each node with respect to a hypothesis is required to determine the level of information a node should hold to accurately predict a change at the sub-network level as well as at the BN level. The selection of the dimension size of the embedding vector takes into account time and space complexity in terms of Big O notation, and hypothesis specifications in terms of a sub-network size involved. In the present disclosure, high-dimensional embedding vectors are considered. The determination of the dimension size of the embedding vector for each node is described in detail, for example, in FIGS. 6 and 7.

The processor 104 is further configured to identify one or more changes in each sub-network by comparing the embedding vector of each node of the set of nodes in the respective sub-network before and after an input action associated with a change in at least one sub-network. After determination of the embedding vector and the dimension size of the embedding vector, the embedding vector of each node of the set of nodes in the respective sub-network is compared before and after of the input action associated with the change in the at least one sub-network. The comparison leads to the identification of the one or more changes happened in each sub-network because of the input action.

In accordance with an embodiment, the input action associated with the change in at least one sub-network is one of: an addition of a new node in the at least one sub-network, or deletion of a node from the at least one sub-network, or creation of an extra edge in the at least one sub-network. For example, in an implementation scenario, the new node is added to the one sub-network then, one or more changes happen in the respective sub-network. The one or more changes may include type of relationships of the new node with each of the set of nodes in the respective sub-network, neighbouring nodes of the new node, type of edges of the new node in the respective sub-network, label of the new node, and the like. Similar is the scenario with the deletion of the node from the at least one sub-network and the creation of the extra edge in the at least one sub-network.

In accordance with an embodiment, the processor 104 is further configured to compare the embedding vector of each node in the set of nodes in each sub-network using a similarity measure. The embedding vector of each node (i.e., node embeddings) are compared before and after of the input action using one of similarity measures, such as Euclidean distance, cosine similarity, or Manhattan distance. In the present disclosure, the Manhattan distance is used for comparison of node embeddings because of high-dimensional data. In high-dimensional spaces, data becomes sparser means a greater number of zeros. In a typical Lk norm represented by Equation (1)

$$x , y ∈ {R}^{d} , k ∈ Z , {L}_{k} ( x , y ) = {\sum }_{i = 1}^{d} ( {( {‖ {x}^{i} - {y}^{i} ‖}^{{k}^{1}} )}^{\frac{1}{k}} ) \qquad ( 1 )$$

As k increases, the Lk norm worsens faster with increasing dimensionality. This means that the L1 distance metric (i.e., the Manhattan distance metric) is the most preferable for high dimensional data. The Manhattan distance represents the sum of absolute differences between coordinates of two points according to Equation (2)

$$distance = {\sum }_{1}^{n} \vert {p}_{i} - {q}_{i} \vert \qquad ( 2 )$$

where p and q are two different embedding vectors, and i is the ith element in each vector. The identified one or more changes in each sub-network comprises topological or behavioural changes in a complex network (e.g., the HBN 110) using the node embeddings and these changes are computed using the Manhattan distance.

The processor 104 is further configured to determine a plurality of scores for each node of the set of nodes of each sub-network based on a pre-defined set of parameters. The pre-defined set of parameters are considered for each node with respect to a hypothesis which determine the role of each node very specific to that hypothesis. The pre-defined set of parameters includes number of hops that is distance from the hypothesis, literature coverage with the hypothesis, topological changes due to the hypothesis, Lagrange Multiplier (LM) score with the hypothesis, and the like. The pre-defined set of parameters are described in detail, for example, in Table 1:

TABLE 1

| | Interpretation | | |
|---|---|---|---|
| Parameter | observed | Formula | Type |
| Distance | The Distance of hops | MATCH | Integer |
| from the | from the hypothesis. | (n: TARGET{Gene_symbol: | |
| hypothesis | If the distance is less | ‘EGFR’}), (m: TARGET | |
| | means, the considered | {Gene_symbol: ‘METAP2’}, | |
| | hypothesis is right | path = shortestPath((n)- | |
| | | [:interacts*]-(m)) | |
| | | RETURN | |
| | | n.Gene_symbol, SIZE([p | |
| | | IN nodes(path) WHERE p: | |
| | | TARGET]) AS count | |
| Literature | If a good number of | query = | Integer |
| coverage | literature coverage | PublicationIndex.search(AND | |
| with the | is obtained with the | (‘EGFR’, ‘METAP2’)) | |
| hypothesis | hypothesis then, | for count in query.count(client): | |
| | the considered | print(count) | |
| | hypothesis is right. | | |
| Topological | The topological | distance = Σ₁ⁿ\|pᵢ − qᵢ\| | Float |
| change due to | change is calculated. | | |
| hypothesis | If the change is large | | |
| | then, the considered | | |
| | hypothesis is right | | |
| LM Score | Higher the LM score | | Float |
| with | means the considered | | |
| hypothesis | hypothesis is right | | |

The processor 104 is further configured to identify the one or more changes in the BN 108 based on the determined plurality of scores for each node of the set of nodes in each sub-network, wherein the plurality of scores is processed at each sub-network and at the BN 108 for identification of the one or more changes in the BN 108. The plurality of scores determined for each node in each sub-network is further processed at the sub-network level as well as at the BN level to identify the one or more changes in the BN 108 (more specifically, in the HBN 110).

In accordance with an embodiment, the processor 104 is further configured to aggregate the plurality of scores determined for each node in each sub-network into a single score to quantify the identified one or more changes in each sub-network and integrate the single score of each node in each sub-network to quantify the identified one or more changes in the BN 108. The plurality of scores determined for each node in each sub-network is aggregated into the single score for quantification of the identified one or more changes in each sub-network. The pre-defined set of parameters is used to prioritize the nodes which are affected considerably by keeping into account the distance of the nodes from the change. Moreover, the co-occurrence of relations between various biological entities are also considered using relevant biomedical publications count. The mathematical expression used for aggregation of the plurality of scores into the single score is represented according to Equation (3)

$$aggregation score = \frac{{d}_{i}^{w}}{{d}_{i}^{w} + {d}_{i}^{b}} \qquad ( 3 )$$

$$where {d}_{i}^{b} = \sqrt{{\sum }_{j = 1}^{N} {( {χ}_{ij} - {χ}_{j}^{b} )}^{2}}$$

$${d}_{i}^{w} = \sqrt{{\sum }_{j = 1}^{N} {( {χ}_{ij} - {χ}_{j}^{w} )}^{2}}$$

and Xⱼ^{b}, Xⱼ^{w} are best and worst alternatives for each criterion calculated by using Equation (4), and Equation (5), respectively.

$${χ}_{j}^{b} = {max}_{i = 1} {χ}_{ij} \qquad ( 4 )$$

$${χ}_{j}^{w} {min}_{i = 1} {χ}_{ij} \qquad ( 5 )$$

Xᵢⱼ is calculated according to Equation (6)

$${χ}_{i j} = {a}_{i j} \times {w}_{j} \qquad ( 6 )$$

aᵢⱼ is normalized data that means each row of data is in between 0 and 1.
wⱼ are weights of the pre-defined set of parameters calculated using D-critic operation.

After computation of the single score for each node at the sub-network level, the computed single score of each node is integrated, firstly at the sub-network level and then, at the BN level, to compute a network mutation score for the BN 108. In order to quantify the identified one or more changes at the BN 108, a vector ν is created according to Equation (7)

$$v ( i ) = {\sum }_{1}^{n} ( mutation score ) ( i ) \qquad ( 7 )$$

where, i belongs to protein set.

The Euclidean distance of the vector ν from an original vector in the dimensional space represents the network mutations score. The network mutations score can be further used to compare changes in biological networks and determine the best and worst among the identified hypotheses.

Thus, the system 102 efficiently quantifies the flow of information and the one or more changes among the number of biological entities of the BN 108 as well as of the HBN 110 and thereby, improves the prediction of disease conditions, therapeutic responses and lead identifications. For quantification of the changes of the BN 108 as well as of the HBN 110, the system 102 is configured to compare and analyse different hypotheses related to therapeutic responses and disease mechanisms. The system 102 is configured to use graph theory and database technologies to process, analyse and visualise the BN 108 and the HBN 110 and derive deeper insights (i.e., indirect relationships among biological entities) using graph projections, embedding vectors and statistical solutions. By virtue of using the graph projections, embedding vectors and statistical solutions, the system 102 is configured to measure the network mutations score of the BN 108 which, become a basis to accept or to negate the hypotheses. The network mutations may also be compared with each other across different hypotheses to check which hypothesis is better. Moreover, the system 102 can be customized according to the nature of the hypothesis. Conventionally, betweenness centrality is used to calculate unweighted shortest paths between all pairs of nodes in a biological network. Each node receives a score based on the number of shortest paths that pass through the node. The nodes lying more frequently on the shortest paths between other nodes have higher betweenness centrality scores. Hence, the node significance is computed by considering only the network structure. However, the system 102 is configured to use the network structure, such as change in the node embeddings before and after removing nodes, etc. as well as consider other parameters, such as publication count, LM score and aggregation score of each node, which indicate the functionality of the node. Thus, the system 102 considers both the network structure as well as the functionality of the node which overcome the limitations of the betweenness centrality.

Moreover, the system 102 may be used to determine significant changes in the BN 108 when a selected molecular entity is added or removed from the BN 108. For example, an experiment may be conducted on whole data which is available form a graphical database having 20205 nodes count and 80817 relationship count. The changes in node embeddings are identified after removing a molecular entity, for example, METAP2. Thereafter, similarity between original node embeddings and the node embeddings obtained after removing METAP2 is computed using Manhattan distance. Additionally, the system 102 may also be used for various purposes, such as target prioritization, drug-drug and target-target combination, drug repurposing, prioritizing disease-associated genes, and the like.

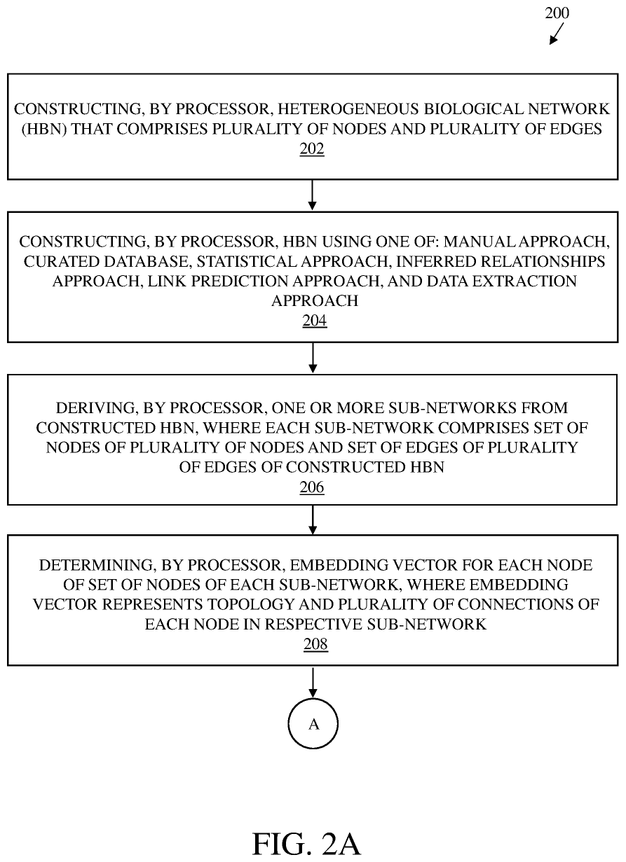

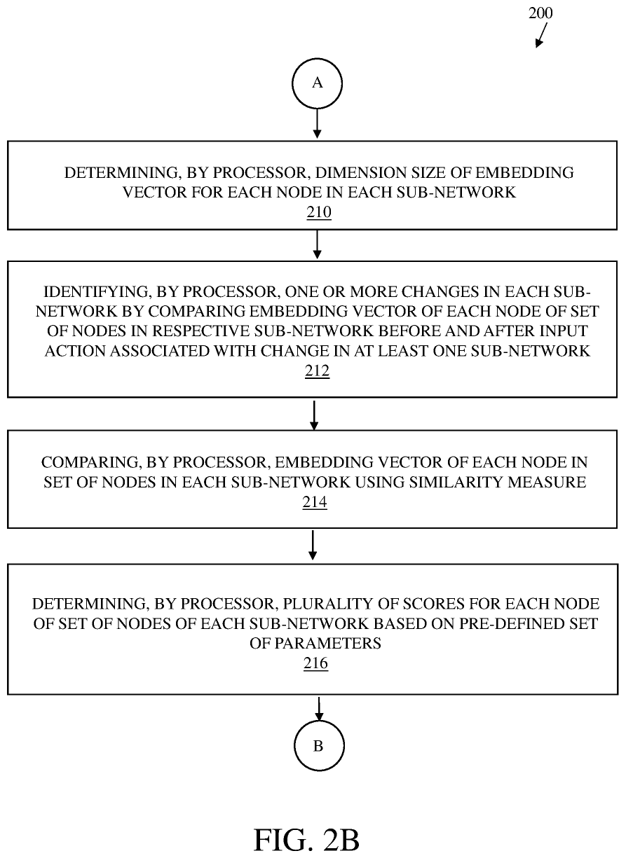

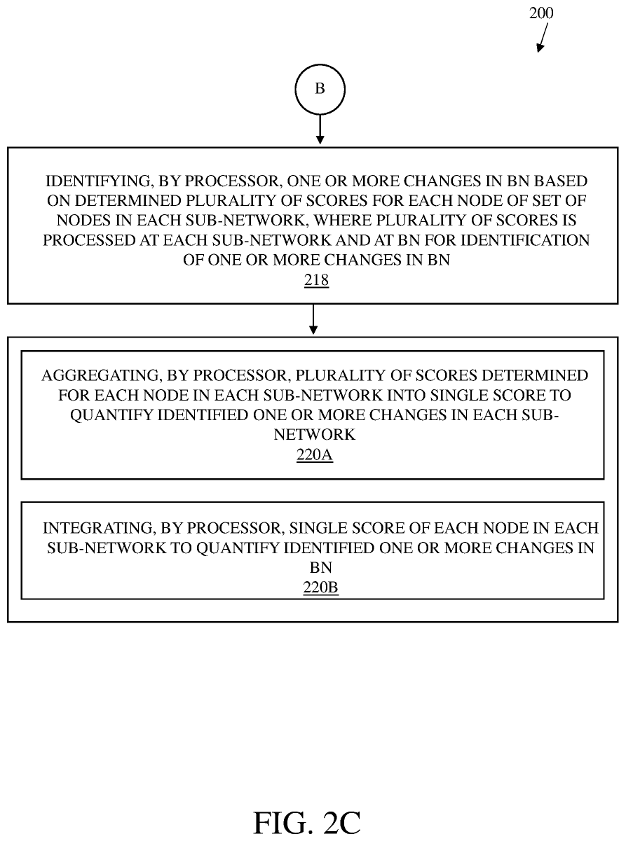

FIGS. 2A to 2C collectively is a flowchart of a method for identifying one or more changes in a Biological Network (BN), in accordance with an embodiment of the present disclosure. FIGS. 2A-2C are described in conjunction with elements from FIG. 1. With reference to FIGS. 2A to 2C, there is shown a flowchart of a method 200 that includes steps 202, 204, 206, 208, 210, 212, 214, 216, 218, 220A, and 220B. The steps 202 to 208 are shown in FIG. 2A, the steps 210, 212, 214, and 216 are shown in FIG. 2B, and the steps 218, 220A, and 220B are shown in FIG. 2C. The method 200 is executed by the processor 104 of the system 102 (of FIG. 1).

There is provided the method 200 for identifying one or more changes in the BN 108. The method 200 is used to quantify information in the BN 108 and identify relevant network mutations of the BN 108 using graphical and database technologies. The network mutations in the BN 108 are used to make decisions about a hypothesis which is based on complex inter-connections of the biological entities which are in-direct relationship with other heterogeneous biological entities.

At step 202, the method 200 comprises constructing, by the processor 104, the HBN 110 that comprises a plurality of nodes and a plurality of edges. The HBN 110 is built with multiple labels of nodes like protein, gene, drug, disease, etc. and relationships like activation, inhibition between various biological entities.

At step 204, the method 200 further comprises constructing, by the processor 104, the HBN 110 using one of: a manual approach, curated database, statistical approach, inferred relationships approach, link prediction approach, and data extraction approach. The HBN 110 is constructed using either machine learning or deep learning algorithms, or by text processing, or using databases, such as DrugBank, and the like.

At step 206, the method 200 further comprises deriving, by the processor 104, one or more sub-networks from the constructed HBN 110, wherein each sub-network comprises a set of nodes of the plurality of nodes and a set of edges of the plurality of edges of the constructed HBN 110. The one or more sub-networks derived from the constructed HBN 110 are required to analyse the direct or indirect relationships among various biological entities in detail. The one or more sub-networks derived from the constructed HBN 110 may include a homogeneous network, a heterogeneous network, a heterogeneous multi-layered network or a combination of aforementioned networks depending on an application scenario.

At step 208, the method 200 further comprises determining, by the processor 104, an embedding vector for each node of the set of nodes of each sub-network, wherein the embedding vector represents a topology and a plurality of connections of each node in a respective sub-network. The embedding vector stores the information about each node of the set of nodes of each sub-network.

Now referring to FIG. 2B, at step 210, the method 200 further comprises determining, by the processor 104, a dimension size of the embedding vector for each node in each sub-network. The determination of the dimension size of the embedding vector for each node with respect to a hypothesis is required to determine the level of information a node should hold to accurately predict a change at the sub-network level as well as at the BN level.

At step 212, the method 200 further comprises identifying, by the processor 104, one or more changes in each sub-network by comparing the embedding vector of each node of the set of nodes in the respective sub-network before and after an input action associated with a change in at least one sub-network. The input action associated with the change in at least one sub-network is one of: an addition of a new node in the at least one sub-network, or deletion of a node from the at least one sub-network, or creation of an extra edge in the at least one sub-network.

At step 214, the method 200 further comprises comparing, by the processor 104, the embedding vector of each node in the set of nodes in each sub-network using a similarity measure. The embedding vector of each node is compared before and after of the input action using one of similarity measures, such as Euclidean distance, cosine similarity, or Manhattan distance, have been described in detail, for example, in FIG. 1.

At step 216, the method 200 further comprises determining, by the processor 104, a plurality of scores for each node of the set of nodes of each sub-network based on a pre-defined set of parameters. The pre-defined set of parameters includes number of hops that is distance from the hypothesis, literature coverage with the hypothesis, topological changes due to the hypothesis, Lagrange Multiplier (LM) score with the hypothesis, and the like.

Now referring to FIG. 2C, at step 218, the method 200 further comprises identifying, by the processor 104, the one or more changes in the BN 108 based on the determined plurality of scores for each node of the set of nodes in each sub-network, wherein the plurality of scores is processed at each sub-network and at the BN 108 for identification of the one or more changes in the BN 108.

At step 220A, the method 200 further comprises aggregating, by the processor 104, the plurality of scores determined for each node in each sub-network into a single score to quantify the identified one or more changes in each sub-network. The plurality of scores determined for each node in each sub-network is aggregated into the single score for quantification of the identified one or more changes in each sub-network, have been described in detail, for example, in FIG. 1.

At step 220B, the method 200 further comprises integrating, by the processor 104, the single score of each node in each sub-network to quantify the identified one or more changes in the BN 108. After computation of the single score for each node at the sub-network level, the computed single score of each node is integrated, firstly at the sub-network level and then, at the BN level, to compute a network mutation score for the BN 108, have been described in detail, for example, in FIG. 1.

The steps 202, 204, 206, 208, 210, 212, 214, 216, 218, 220A, and 220B are only illustrative and other alternatives can also be provided where one or more steps are added, one or more steps are removed, or one or more steps are provided in a different sequence without departing from the scope of the claims herein.

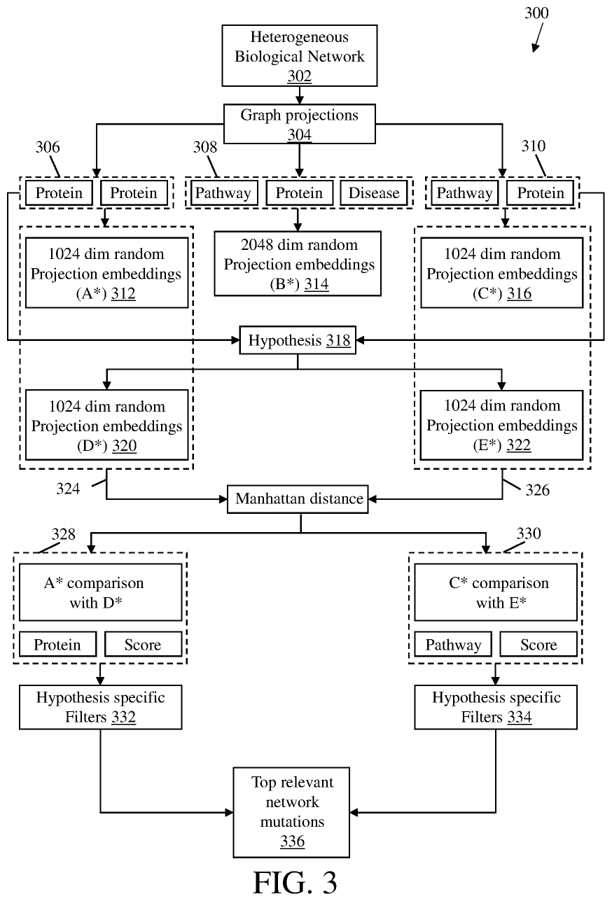

FIG. 3 is a flowchart to identify network mutations in a heterogeneous biological network, in accordance with an embodiment of the present disclosure. FIG. 3 is described in conjunction with elements from FIGS. 1, and 2A-2C. With reference to FIG. 3, there is shown a flowchart 300 to identify network mutations in a heterogeneous biological network 302. The flowchart 300 includes a series of operations 304 to 336.

The heterogeneous biological network 302 corresponds to the HBN 110 (of FIG. 1) which includes a plurality of nodes representing a number of biological entities and a plurality of edges representing a number of relationships among the number of biological entities.

At operation 304, the heterogeneous biological network 302 is presented in a graphical representation by use of, for example, neo4j technology.

At operations 306, 308, and 310, one or more sub-networks are derived from the heterogeneous biological network 302. For example, at the operation 306, a first sub-network (e.g., a Protein-Protein sub-network), at the operation 308, a second sub-network (e.g., a Pathway-Protein-Disease sub-network), and at the operation 310, a third sub-network (e.g., a Pathway-Protein sub-network) are derived from the heterogeneous biological network 302.

At operation 312, node embeddings for each node of the first sub-network are generated. For example, random projection embeddings of 1024 dimensions (may also be represented as A*) are generated for each node of the first sub-network (i.e., the Protein-Protein sub-network). Similarly, at operations 314 and 316, node embeddings for each node of the second sub-network and the third sub-network are generated, respectively. For example, random projection embeddings of 2048 dimensions (may also be represented as B*) are generated for each node of the second sub-network (i.e., the Pathway-Protein-Disease sub-network) at the operation 314. Similarly, random projection embeddings of 1024 dimensions (may also be represented as C*) are generated for each node of the third sub-network (i.e., the Pathway-Protein sub-network) at the operation 316.

At operation 318, a hypothesis is used at as an input to the first sub-network (i.e., the Protein-Protein sub-network) and the third sub-network (i.e., the Pathway-Protein sub-network).

At operations 320 and 322, node embeddings for each node of the first sub-network and the third sub-network are regenerated, respectively, after inputting the hypothesis. For example, random projection embeddings of 1024 dimensions (may also be represented as D*) are regenerated for each node of the first sub-network (i.e., the Protein-Protein sub-network) at the operation 320. Similarly, random projection embeddings of 1024 dimensions (may also be represented as E*) are regenerated for each node of the third sub-network (i.e., the Pathway-Protein sub-network) at the operation 322.

At operation 324, node embeddings (i.e., A* and D*) for each node of the first sub-network (i.e., the Protein-Protein sub-network) are compared before and after the input of the hypothesis using Manhattan distance.

At operation 326, node embeddings (i.e., C* and E*) for each node of the third sub-network (i.e., the Pathway-Protein sub-network) are compared before and after the input of the hypothesis using Manhattan distance.

At operation 328, one or more changes are identified in the first sub-network (i.e., the Protein-Protein sub-network) by comparison of A* and D* and a score is assigned to each node of the first sub-network.

At operation 330, one or more changes are identified in the third sub-network (i.e., the Pathway-Protein sub-network) by comparison of C* and E* and a score is assigned to each node of the third sub-network.

At operation 332, specific filters associated with the hypothesis are applied on the score assigned to each node of the first sub-network (i.e., the Protein-Protein sub-network) to compute relevant network mutations of the first sub-network.

At operation 334, specific filters associated with the hypothesis are applied on the score assigned to each node of the third sub-network (i.e., the Pathway-Protein sub-network) to compute relevant network mutations of the third sub-network.

At operation 336, the relevant network mutations of the first sub-network and the third sub-network are analysed for computations of relevant network mutations of the heterogeneous biological network 302.

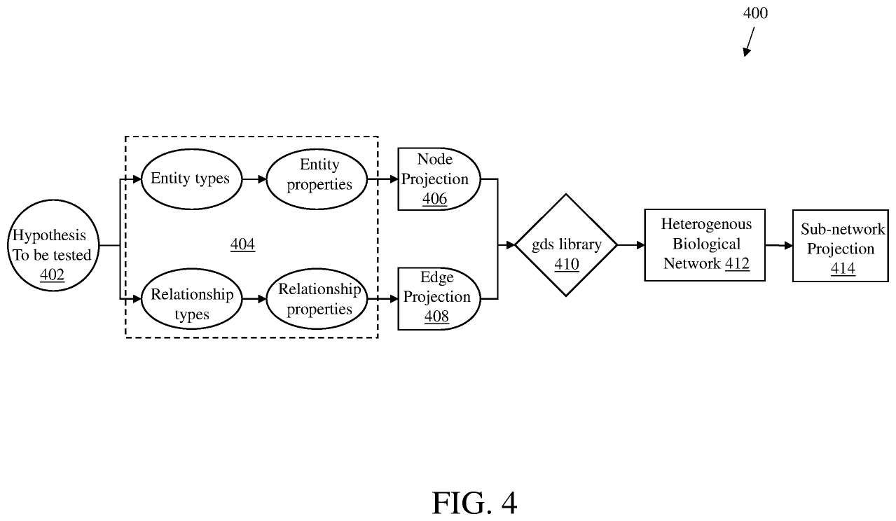

FIG. 4 illustrates derivation of one or more sub-networks from a heterogeneous biological network, in accordance with an embodiment of the present disclosure. FIG. 4 is described in conjunction with elements from FIGS. 1, 2A-2C, and 3. With reference to FIG. 4, there is shown an illustration 400 which depicts derivation of one or more sub-networks from a heterogeneous biological network. There is further shown a series of operations 402 to 410.

At operation 402, a hypothesis which requires testing is used as an input.

At operation 404, various types of biological entities as well as their relationship types are identified. Furthermore, various properties of the biological entities as well as their relationship properties are identified.

At operation 406, after identification of the biological entities and their associated properties, a number of nodes are projected.

At operation 408, after identification of relationship types of the biological entities and the relationship properties of the biological entities, a number of edges are projected.

At operation 410, the projected nodes and the projected edges are used as an input to a Graph Data Science (GDS) library (e.g., a neo4j GDS library) in order to generate a heterogeneous biological network 412. The heterogeneous biological network 412 corresponds to the HBN 110, of FIG. 1. Furthermore, one or more sub-networks 414 are derived from the heterogeneous biological network 412. The one or more sub-networks 414 are used to perform a fast and precise analysis depending on a use case and derive in-depth insights relevant to the focused hypothesis. Moreover, embedding vectors for each node of each sub-network and statistical techniques are used either to accept or to negate the hypothesis used as the input.

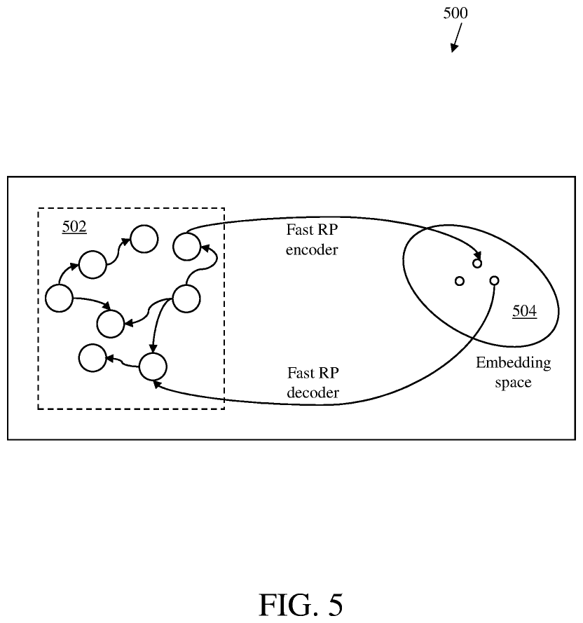

FIG. 5 illustrates transformation of a heterogeneous biological network into an embedding space, in accordance with an embodiment of the present disclosure. FIG. 5 is described in conjunction with elements from FIGS. 1, 2A-2C, 3, and 4. With reference to FIG. 5, there is shown an illustration 500 that depicts transformation of a heterogeneous biological network 502 into an embedding space 504.

The heterogeneous biological network 502 corresponds to the HBN 110, of FIG. 1. After derivation of one or more sub-networks from the heterogeneous biological network 502, information about each node of each sub-network is stored in a high-dimensional embedding space (i.e., the embedding space 504) by creating a vector which depicts topology and connections of each node in a respective sub-network. Each node is transformed into the embedding space 504 by use of a Fast Random Projection (Fast RP) encoder. The node embeddings of different lengths are created depending on a use case and the best suitable vector size is identified. The embedding space 504 can be de-transformed into a respective node by use of a Fast RP decoder.

FIG. 6 is a graphical representation that depicts average running time to generate node embeddings of different lengths, in accordance with an embodiment of the present disclosure. FIG. 6 is described in conjunction with elements from FIGS. 1, 2A-2C, 3, 4 and 5. With reference to FIG. 6, there is shown a graphical representation 600 that includes a X-axis 602 and a Y-axis 604. The X-axis 602 represents a node count and the Y-axis 604 represents time in seconds. The graphical representation 600 depicts average running time (i.e., time complexity) of node embeddings of different embedding sizes ranging from a dimension size of 16 to a dimension size of 2048 which, is further used for time and complexity analysis.

FIG. 7 is a bar graph representation that depicts number of embeddings versus count of embeddings having all zero elements, in accordance with an embodiment of the present disclosure. FIG. 7 is described in conjunction with elements from FIGS. 1, 2A-2C, 3, 4, 5 and 6. With reference to FIG. 7, there is shown a bar graph representation 700 that depicts number of embeddings versus count of embeddings having all zero elements. With reference to FIG. 7, there is shown a X-axis 702 that represents number of embeddings and a Y-axis 704 that represents count of embeddings having all zero elements. In the bar graph representation 700, three bars are shown, for example, a first bar 706, a second bar 708 and a third bar 710.

In order to determine an appropriate dimension size of a node embedding for a hypothesis, a count of zero and nonzero elements are considered in each node embedding and then, sparsity is calculated according to Equation (8)

$$\frac{{\sum }_{i = 1}^{n} [ 1 if i not 0 ]}{n} \qquad ( 8 )$$

The node embeddings of different sizes are considered, such as the first bar 706 represents a node embedding of size 128. Similarly, the second bar 708 and the third bar 710 represent node embeddings of sizes 256 and 1024, respectively. Thereafter, sparsity is calculated for each of the node embeddings. It is observed that as the size of node embeddings increases, the number of non-zero embeddings also increases which is used to store complex network topological features on a node. For this reason, node embeddings of large size are considered.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural. The word “exemplary” is used herein to mean “serving as an example, instance or illustration”. Any embodiment described as “exemplary” is not necessarily to be construed as preferred or advantageous over other embodiments and/or to exclude the incorporation of features from other embodiments. The word “optionally” is used herein to mean “is provided in some embodiments and not provided in other embodiments”. It is appreciated that certain features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable combination or as suitable in any other described embodiment of the disclosure.

1. A system for identifying one or more changes in a Biological Network (BN), comprising:
a processor configured to:
construct a Heterogeneous Biological Network (HBN) that comprises a plurality of nodes and a plurality of edges;
derive one or more sub-networks from the constructed HBN, wherein each sub-network comprises a set of nodes of the plurality of nodes and a set of edges of the plurality of edges of the constructed HBN;
determine an embedding vector for each node of the set of nodes of each sub-network, wherein the embedding vector represents a topology and a plurality of connections of each node in a respective sub-network;
identify one or more changes in each sub-network by comparing the embedding vector of each node of the set of nodes in the respective sub-network before and after an input action associated with a change in at least one sub-network;
determine a plurality of scores for each node of the set of nodes of each sub-network based on a pre-defined set of parameters; and
identify the one or more changes in the BN based on the determined plurality of scores for each node of the set of nodes in each sub-network, wherein the plurality of scores is processed at each sub-network and at the BN for identification of the one or more changes in the BN.

2. The system according to claim 1, wherein the processor is further configured to:
aggregate the plurality of scores determined for each node in each sub-network into a single score to quantify the identified one or more changes in each sub-network; and
integrate the single score of each node in each sub-network to quantify the identified one or more changes in the BN.

3. The system according to claim 1, wherein the processor is further configured to construct the HBN using one of: a manual approach, curated database, statistical approach, inferred relationships approach, link prediction approach, and data extraction approach.

4. The system according to claim 1, wherein the one or more sub-networks comprises a homogeneous network, a heterogeneous network, a heterogeneous multi-layered network, or a combination thereof.

5. The system according to claim 1, wherein the processor is further configured to determine a dimension size of the embedding vector for each node in each sub-network.

6. The system according to claim 1, wherein the input action associated with the change in at least one sub-network is one of: an addition of a new node in the at least one sub-network, or deletion of a node from the at least one sub-network, or creation of an extra edge in the at least one sub-network.

7. The system according to claim 1, wherein the processor is further configured to compare the embedding vector of each node in the set of nodes in each sub-network using a similarity measure.

8. A method for identifying one or more changes in a Biological Network (BN), comprises:
constructing, by a processor, a Heterogeneous Biological Network (HBN) that comprises a plurality of nodes and a plurality of edges;
deriving, by the processor, one or more sub-networks from the constructed HBN, wherein each sub-network comprises a set of nodes of the plurality of nodes and a set of edges of the plurality of edges of the constructed HBN;
determining, by the processor, an embedding vector for each node of the set of nodes of each sub-network, wherein the embedding vector represents a topology and a plurality of connections of each node in a respective sub-network;
identifying, by the processor, one or more changes in each sub-network by comparing the embedding vector of each node of the set of nodes in the respective sub-network before and after an input action associated with a change in at least one sub-network;
determining, by the processor, a plurality of scores for each node of the set of nodes of each sub-network based on a pre-defined set of parameters; and
identifying, by the processor, the one or more changes in the BN based on the determined plurality of scores for each node of the set of nodes in each sub-network, wherein the plurality of scores is processed at each sub-network and at the BN for identification of the one or more changes in the BN.

9. The method according to claim 8, wherein the method further comprises:
aggregating, by the processor, the plurality of scores determined for each node in each sub-network into a single score to quantify the identified one or more changes in each sub-network; and
integrating, by the processor, the single score of each node in each sub-network to quantify the identified one or more changes in the BN.

10. The method according to claim 8, wherein the method further comprises constructing, by the processor, the HBN using one of: a manual approach, curated database, statistical approach, inferred relationships approach, link prediction approach, and data extraction approach.

11. The method according to claim 8, wherein the one or more sub-networks comprises a homogeneous network, a heterogeneous network, a heterogeneous multi-layered network, or a combination thereof.

12. The method according to claim 8, wherein the method further comprises determining, by the processor, a dimension size of the embedding vector for each node in each sub-network.

13. The method according to claim 8, wherein the input action associated with the change in at least one sub-network is one of: an addition of a new node in the at least one sub-network, or deletion of a node from the at least one sub-network, or creation of an extra edge in the at least one sub-network.

14. The method according to claim 8, wherein the method further comprises comparing, by the processor, the embedding vector of each node in the set of nodes in each sub-network using a similarity measure.
